(19) United States
(12) Patent Application Publication
(10) Pub. No.: US 2006/0188529 A1
(43) Pub. Date: Aug. 24, 2006

(54) Stable compositions of fenofibrate with fatty acid esters

(76) Inventors: George Bobotas, Tarpon Springs, FL (US); Roelof M.L. Rongen, Caliton, NJ (US); Abdel Fawzy, Somerville, NJ (US)

Correspondence Address: ARENT FOX PLLC, 1050 CONNECTICUT AVENUE, N.W., SUITE 400, WASHINGTON, DC, 20036, US

(21) Appl. No.: 11/293,496

(22) Filed: Dec. 5, 2005

Related U.S. Application Data
(60) Provisional application No. 60/633,126, filed on Dec. 6, 2004.

(51) Int. Cl. A61K 31/192; A61K 9/00

(52) U.S. Cl. 424/400; 514/571

(57) ABSTRACT

A pharmaceutical composition in unit dose form of fenofibrate and a solvent system of fatty acid esters, wherein the fenofibrate is substantially dissolved in the solvent system.

This application claims priority from provisional application Ser. No. 60/633,126, filed Dec. 6, 2004. The disclosure of the provisional application is hereby incorporated by reference in its entirety.

### FIELD OF THE INVENTION

The present invention relates to a stable fenofibrate compositions comprising fenofibrate and fatty acid esters, in which the fenofibrate is solubilized. The compositions are useful for the treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes.

### BACKGROUND OF THE INVENTION

In humans, high levels of total cholesterol, low-density lipoproteins (LDL), and apolipoprotein B (a membrane complex for LDL-C) promote atherosclerosis. These high levels also promote lower levels of high-density lipoproteins (HDL), and apolipoprotein A (HDL transport complex), which are also associated with the development of atherosclerosis. Cardiovascular morbidity and mortality also vary directly with the level of total cholesterol and LDL and inversely with the level of HDL.

Agents such as fibrates have typically been used in patients to decrease lipoproteins rich in triglycerides, to increase HDL cholesterol and to decrease atherogenic-dense LDL. Fibrates have also been used to treat post-myocardial infarction (MI) and adult endogenous hyperlipidemias of hypercholesterolemias and of hypertriglyceridemias.

Fenofibrate, or 2-[4-(4-chlorobenzoyl)phenoxy]-2-methyl-propanoic acid, 1-methylethyl ester, has been known for many years as a medicinal active principle because of its efficacy in lowering blood triglyceride and cholesterol levels. A treatment of 40 mg to 300 mg of fenofibrate per day enables a 20% to 25% reduction of cholesterolemia and a 40% to 50% reduction of triglyceridemia.

Fenofibrate is, however, very poorly soluble in water and its absorption in the digestive tract is limited. Various approaches have been explored in order to increase the rate of solubilization of fenofibrate, including micronization of the active principle, addition of surfactants, and co-micronization of fenofibrate with a surfactant. Examples of attempts to increase the rate of solubilization of fenofibrate may be found in U.S. Pat. No. 4,895,726, U.S. Pat. No. 6,074,670, U.S. Pat. No. 6,277,405, U.S. Pat. No. 6,589,552 and U.S. Pat. No. 6,652,881, the contents of all of these patents are incorporated in their entirety herein by reference.

U.S. Pat. Nos. 6,096,338, 6,267,985, 6,667,064, and 6,720,001, U.S. Pat. Appl. Pub. Nos. 2003/0082215 and 2004/0052824, WO 99/29300, and WO 2001/021154, the contents of all of these documents are incorporated in their entirety herein by reference, disclose compositions, carrier systems and oil-in-water emulsions containing digestible oils or triglycerides with an active ingredient, such as fenofibrate. Specific combinations of fenofibrate and fatty acid esters are not disclosed. Further, these compositions require surfactants to solubilize the fenofibrate. For example, U.S. Pat. No. 6,284,268, the contents of which are incorporated in their entirety herein by reference, is directed to self-emulsifying pre-concentrate pharmaceutical compositions capable of forming oil-in-water microemulsions or emulsions upon dilution with an aqueous solution. The patent describes an omega-3 fatty acid oil and a poorly water soluble therapeutic agent, such as a cyclosporin or fenofibrate. The formulations in this patent, however, use a large amount of solubilizers such as surfactant (generally higher than 50% w/w, based on the weight of the solvent system) to achieve the self-emulsifying compositions.

U.S. Pat. Nos. 5,645,856 and 6,096,338 are directed to compositions and methods for improving the in vivo bioavailability of a hydrophobic drug. The drug is dispersed or dissolved in a digestible oil containing a hydrophilic surfactant that substantially inhibits the in vivo lipolysis of the digestible oil. The composition also includes a lipophilic surfactant capable of reducing the inhibitory effect of the hydrophilic surfactant.

U.S. Pat. No. 5,827,536 discloses soluble fenofibrate pharmaceutical dosage formulations exhibiting improved bioavailability after oral administration. The formulations contain fenofibrate as a solution in a solubilizing agent of diethylene glycol monoethyl ether.

Nigon et al. disclose that the consumption of a spread enriched with a mixture of esters of sitosterol, campesterol and stigmasterol, at low doses, is effective in lowering plasma total cholesterol and LDL-C levels in hypercholesterolemic patients at high cardiovascular risk. Nigon et al., Clin. Chem. Lab. Med., 39(7):634-40 (2001). Nigon et al. further discloses that plasma total cholesterol and LDL-C were significantly lower in a subgroup of patients treated with fibrates, after consumption of the phytosterol ester-enriched spread.

More recently, Yeganeh et al. disclosed that a combination of dietary phytosterols and niacin or fenofibrate impacts lipoprotein profile and atherogenesis in apo EKO mice. Yeganeh et al., J. Nutritional Biochemistry 16:222-28 (2005). In particular, it was shown that the addition of fenofibrate to phytosterols synergistically increased plasma total cholesterol levels by >50% and decreased HDL cholesterol concentrations by 50%. The combination of fenofibrate to phytosterols had no effect on plasma triglyceride levels. Yeganeh et al. concluded that patients who are taking fenofibrate may not additionally benefit from phytosterol-enriched food products.

The inventors have unexpectedly found that fenofibrate is completely soluble in fatty acid esters, with minimal or no use of surfactants or other solubilizing agents or techniques. Compositions in which the majority of components are fenofibrate and fatty acid esters have the significant advantage of delivering more fenofibrate to the patient in a smaller pill or tablet than traditional compositions, which require large amounts of surfactants or other solubilizing agents.

### SUMMARY OF THE INVENTION

One aspect of the invention is directed to compositions of fenofibrate and fatty acid esters in which the fenofibrate is essentially completely dissolved.

A second aspect of the invention is directed to compositions of fenofibrate and fatty acid esters that do not require surfactants or other solubilizing agents or techniques, such as micronization, in order to solubilize the fenofibrate.

A third aspect of the invention is directed to compositions of fenofibrate and fatty acid C₁ to C₁₅ esters.

A fourth aspect of the invention is directed to compositions of fenofibrate and fatty acid C₁ to C₁₅ esters, wherein the fatty acid C₁ to C₁₅ esters are also “active” components.

A fifth aspect of the invention is directed to compositions of fenofibrate and C₁ to C₁₅ esters of omega-3, omega-5, omega-6, omega-7, and omega-9 fatty acids.

A sixth aspect of the invention is directed to compositions of fenofibrate and C₁ to C₁₅ esters of one or more sterols or stanols.

A seventh aspect of the invention is directed to oral dosage forms comprising compositions of fenofibrate and C₁ to C₁₅ esters of fatty acids.

An eighth aspect of the invention is directed to treatment of diseases by administering compositions of fenofibrate and C₁ to C₁₅ esters of fatty acids.

Other novel features and advantages of the present invention will become more apparent to those skilled in the art upon examination of the following or upon learning by practice of the invention.

### DETAILED DESCRIPTION OF THE INVENTION

The fenofibrate is essentially completely solubilized in the fatty acid esters, which allows for improved administration of fenofibrate. In accordance with the present invention, at least 90% w/w of the fenofibrate is dissolved in the fatty acid ester, preferably at least 95% w/w, and more preferably at least 98% w/w. The dosage form is stable at room temperature (about 23° C. to 27° C.) for a period of at least one month, preferably at least six months, more preferably at least one year, and most preferably at least two years. By “stable”, applicants mean that the solubilized fenofibrate does not come out of solution to any appreciable degree, for example, in amounts of less than 10%, preferably less than 5%.

A combination product comprises an amount of fenofibrate and an amount of fatty acid esters that together are therapeutically effective. The present invention also provides a novel treatment method comprising the administration of fenofibrate in a combination product for the treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes.

If the fatty acid ester is itself an “active” ingredient, an effect greater than any expected combined or additive effect of the two alone is achieved. Thus, the combined treatment of fenofibrate along with another active ingredient through the novel combination product of the present invention, allows increased effectiveness with standard dosages or maintained effectiveness with reduced dosages of the two active ingredients. The side effects are also potentially reduced as a result of the lower dosage amount.

Because of the increased pharmaceutical effect from the treatment of a patient with the combination of active ingredients, the typical dosages of these active ingredients allows for a more effective treatment. In another embodiment, the dosage and accompanying side effects may be reduced while still maintaining an effective treatment. In a third embodiment, the reduced side effects allow for an increase in the amount of fenofibrate above the typical dosages known in the art. Preferred embodiments include the administration of 300 mg or less of fenofibrate, preferably 200 mg or less, more preferably 160 mg or less, even more preferably 140 mg or less, most preferably 130 mg or less.

Any fatty acid ester can be used in the present invention. In one embodiment, either the acid portion or the alcohol portion of the fatty acid ester is selected from a C₁ to C₁₅ group, preferably a C₁ to C₆ group, and more preferably a C₁ to C₄ group. In other embodiments, the fatty acid ester is selected from a methyl ester, n-propyl ester, iso-propyl ester, n-butyl ester, iso-butyl ester, sec-butyl ester, and ter-butyl ester. In a preferred embodiment, the fatty acid ester is an ethyl ester. The esters may be linear, branched, saturated, unsaturated, or polyunsaturated, and may be modified with functional groups including halo, ester, ether, keto, amino, nitrile, carboxy, imino, thio, oxo, cyano, thiocyano, and nitro. The alcohol can be a primary, secondary or tertiary alcohol.

In an embodiment of the present invention, the fatty acid ester can be another “active” such as omega-3, omega-5, omega-6, omega-7, and omega-9 fatty acid esters, as well as their derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof.

Examples of omega-3 fatty acids that can be used as the acid part of their respective esters include, but are not limited to, eicosapentaenoic acid (EPA), docosahexaenoic acid (DHA), and α-linolenic acid. Examples of omaga-5 fatty acids include, but are not limited to, myristoleic acid. Examples of omega-6 fatty acids include, but are not limited to, linoleic acid, gamma-linolenic acid, dihomogammalinolenic acid (DGLA), arachidonic acid, docosadienoic acid, and docosatetraenoic acid. Examples of omega-7 fatty acids include, but are not limited to, palmitoleic acid, heptadecenoic acid, vaccenic acid, and rumenic acid. Examples of omega-9 fatty acids include, but are not limited to, oleic acid and eicosenoic acid.

In another embodiment of the present invention, the fatty acid ester can be another “active” such as sterol or stanol esters, or pharmaceutically acceptable derivatives, conjugates, precursors or salts thereof, or mixtures thereof. The present invention may incorporate now known or future known sterols or stanols in an amount generally recognized as safe. For example, in some embodiments of the present invention the sterol may include one or more of sitosterol, campesterol, stigmasterol, avenasterol, brassicasterol, ergosterol, and lanosterol. In other embodiments of the present invention the stanol may include one or more of cholestanol, sitostanol, campestanol, stigmastanol, avenastanol, brassicastanol, ergostanol, and lanostanol. In preferred embodiments, the sterol is sitosterol. In other preferred embodiments the stanol is sitostanol.

The fatty acid esters can be present in an amount from about 350 mg to about 10 grams, more preferably about 500 mg to about 6 grams, and most preferably from about 750 mg to about 3 grams. This amount may be in one or more dosage forms, preferably one dosage form.

The fenofibrate may be dissolved in the fatty acid esters with or without the use of heat, preferably without heating.

The fenofibrate and fatty acid esters may be administered in a capsule, a tablet, a powder that can be dispersed in a beverage, or another solid oral dosage form, a liquid, a soft gel capsule or other convenient dosage form such as oral liquid in a capsule, as known in the art. In some embodiments, the capsule comprises a hard gelatin. The product may also be contained in a liquid suitable for injection or infusion.

The fenofibrate and fatty acid esters may also be administered with a combination of one or more non-active pharmaceutical ingredients (also known generally herein as “excipients”), as common in the art. For example, stabilizers may be employed to avoid the formation of fenofibrate crystals during handling or storage. Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthum gum.

Excipients include surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The product is aided by the solubility of the fenofibrate in the fatty acid esters. Thus, the product does not require high amounts of solubilizers, such as surfactants, cosolvents, oils or combinations thereof. Preferably, the active ingredients are administered without the use of large amounts of solubilizers (other than the fatty acid esters). In preferred embodiments, if present at all, solubilizers other than the fatty acid esters are present in amounts of less than 50% w/w based on the total weight of the solvent system in the dosage form(s), preferably less than 40%, more preferably less than 30%, even more preferably less than 20%, still more preferably less than 10% and most preferably less than 5%. In some embodiments, the solvent system contains no solubilizers other than the fatty acid esters. As used herein, “solvent system” includes the fatty acid esters. In other preferred embodiments, the weight ratio of fatty acid esters to other solubilizer is at least 0.5 to 1, more preferably at least 1 to 1, even more preferably at least 5 to 1, and most preferably at least 10 to 1.

In other preferred embodiments, if present at all, the amount of hydrophilic solvent used in the solvent system is less than 20% w/w based on the total weight of the solvent system in the dosage form(s), more preferably less than 10%, and most preferably less than 5%. In certain embodiments, the amount of hydrophilic solvent used in the solvent system is between 1 and 10% w/w.

Preferably, the fenofibrate is substantially dissolved (i.e., less than 10%, preferably less than 5% remains undissolved in the solvent system). Most preferably, the fenofibrate is essentially completely dissolved (i.e., less than 2% remains undissolved in the solvent system).

In one embodiment of the present invention, fenofibrate can be present in an amount from about 8 mg to 400 mg, more preferably from about 20 mg to about 300 mg, and most preferably from about 30 mg to about 160 mg. The starting material is preferably crystalline fenofibrate that has not been micronized or exposed to other mechanical techniques. In a preferred embodiment, fenofibrate having a mean particle size of at least 25 μm, preferably at least 50 μm, is dissolved in the fatty acid esters. Preferably, there is no particle size specification requirement for the fenofibrate.

The fenofibrate amount may be in one or more dosage forms, preferably one dosage form. In another embodiment, the fenofibrate is present, in a separate or combined dosage form, in a ratio of about 5 mg to 400 mg, preferably about 25 mg to 200 mg, per gram of fatty acid ester. The daily dosages of fenofibrate and fatty acid esters can be administered together or singly in from 1 to 10 individual dosage forms each, or 1 to 10 combined dosage forms, with the desired number of dosage forms taken 1 to 4 times a day.

Any undesirable side effects may be reduced as a result of the lower dosage amount and the reduction in excipients (e.g., surfactants).

The present invention also includes a method of making a pharmaceutical composition, comprising providing crystalline fenofibrate that has not been micronized or exposed to other mechanical techniques, and substantially dissolving the fenofibrate in a solvent system comprising fatty acid esters.

All references cited herein are incorporated by reference in their entirety.

We claim:

1. A pharmaceutical composition in unit dose form, comprising fenofibrate and a solvent system comprising fatty acid esters, wherein the fenofibrate is substantially dissolved in the solvent system.

2. The pharmaceutical composition of claim 1, wherein the fenofibrate is essentially completely dissolved in the solvent system.

3. The pharmaceutical composition of claim 1, wherein the pharmaceutical composition is stable for at least six months at room temperature.

4. The pharmaceutical composition of claim 1, wherein either the acid portion or the alcohol portion of the fatty acid esters comprises a C₁ to C₁₅ group.

5. The pharmaceutical composition of claim 1, wherein either the acid portion or the alcohol portion of the fatty acid esters comprises a C₁ to C₆ group.

6. The pharmaceutical composition of claim 1, wherein either the acid portion or the alcohol portion of the fatty acid esters comprises a C₁ to C₄ group.

7. The pharmaceutical composition of claim 1, wherein the fatty acid esters comprise omega-3, omega-5, omega-6, omega-7, or omega-9 fatty acid esters or pharmaceutically acceptable derivatives, conjugates, precursors or salts thereof, or mixtures thereof.

8. The pharmaceutical composition of claim 1, wherein the fatty acid esters comprise sterol or stanol fatty acid esters or pharmaceutically acceptable derivatives, conjugates, precursors or salts thereof, or mixtures thereof.

9. The pharmaceutical composition of claim 1, wherein the solvent system contains less than 50% w/w, based on the total weight of the solvent system, of at least one solubilizer other than the fatty acid esters.

10. The pharmaceutical composition of claim 1, wherein the solvent system consists of the fatty acid esters.

11. The pharmaceutical composition of claim 1, wherein the solvent system further comprises at least one solubilizer other than the fatty acid esters in a weight ratio of fatty acid esters to solubilizer of at least 0.5 to 1.

12. The pharmaceutical composition of claim 1, wherein the solvent system contains less than 20% w/w, based on the total weight of the solvent system, of at least one hydrophilic solvent.

13. The pharmaceutical composition of claim 1, wherein the fenofibrate is crystalline fenofibrate that has not been micronized or exposed to other mechanical techniques.

14. The pharmaceutical composition of claim 1, wherein the fenofibrate has a mean particle size of at least 25 μm.

15. A method of making a pharmaceutical composition, comprising providing crystalline fenofibrate that has not been micronized or exposed to other mechanical techniques, and substantially dissolving the fenofibrate in a solvent system comprising fatty acid esters.

16. The method of claim 15, wherein the fenofibrate has a mean particle size of at least 25 μm.

17. The method of claim 15, wherein the fenofibrate is essentially completely dissolved in the solvent system.

18. The method of claim 15, wherein the fatty acid esters comprise omega-3, omega-5, omega-6, omega-7, or omega-9 fatty acid esters or pharmaceutically acceptable derivatives, conjugates, precursors or salts thereof, or mixtures thereof.

19. The method of claim 15, wherein the fatty acid esters comprise sterol or stanol fatty acid esters or pharmaceutically acceptable derivatives, conjugates, precursors or salts thereof, or mixtures thereof.
